(19) United States
(12) Patent Application Publication
(10) Pub. No.: US 2023/0049050 A1
(43) Pub. Date: Feb. 16, 2023

(54) EMULSIONS OF BIOACTIVE LIPOPHILIC COMPOUNDS INCLUDING OILS

(71) Applicants: National Research Council of Canada, Ottawa (CA); Next Remedies Inc., Toronto (CA)

(72) Inventors: Anja Vogt, Charlottetown (CA); Elia G. Martin, Charlottetown (CA); Bob Chapman, Charlottetown (CA); Gen Wang, Charlottetown (CA); Jose Gabriel Gomes Jardim, Marina Del Rey, CA (US)

(21) Appl. No.: 17/787,467

(22) PCT Filed: Dec. 17, 2020
(86) PCT No.: PCT/CA2020/051739

§ 371 Date: Jun. 20, 2022

Related U.S. Application Data
(60) Provisional application No. 62/949,647, filed on Dec. 18, 2019.

(51) Int. Cl. A61K 47/22; A61K 31/122; A61K 31/075; A61K 47/10; A61K 9/107; A61K 47/44; A23L 33/115; A23L 2/52

(52) U.S. Cl. CPC A61K 47/22; A61K 31/122; A61K 31/075; A61K 47/10; A61K 9/107; A61K 47/44; A23L 33/115; A23L 2/52; A23V 2002/00

(57) ABSTRACT

The present application relates to compositions corn-sing a bioactive lipophilic compound, an oil and at least one solubilizing agent that is a compound of Formula (I), to emulsions comprising such compositions and to uses thereof. The present application also relates to methods of preparing such emulsions. The methods comprise heating such compositions to form a homogeneous melt and combining the homogeneous melt with water to obtain the emulsion.

### CROSS-REFERENCE TO RELATED APPLICATIONS

The present application claims the benefit of priority from co-pending U.S. provisional application No. 62/949,647 filed on Dec. 18, 2019, the contents of which are incorporated herein by reference in their entirety.

### FIELD

The present application relates, for example, to compositions comprising a bioactive lipophilic compound, a solubilizing agent and an oil, to emulsions comprising such compositions and to uses thereof.

### BACKGROUND

The market for natural health products (NHPs), functional foods and supplements has been growing steadily and exceeded 300B globally in 2010. New technology is desirable to meet emerging consumer demands such as new delivery formats for example, alternatives to swallowing pills. Most functional ingredients used in NHPs are not water soluble, therefore their delivery mechanism is commonly in the form of a pill, which may not be suitable for a growing percentage of the population, for example, the elderly.

U.S. Pat. No. 6,045,826 discloses water-soluble compositions that comprise a lipophilic compound and a solubilizing agent. The solubilizing agent can be polyoxyethanyl-α-tocopheryl sebacate (PTS). PTS has been used, for example, to solubilize lipophilic compounds and bioactives like coenzyme Q₁₀ (CoQ₁₀) and nutritional oils. It has obtained GRAS status in the United States and is registered in the Health Canada Natural Products Ingredients Database (NHPID). However, no commercial products comprising PTS have reached the market. For the commercialization of a food product, beverage product and/or a supplement, an emulsion desirably is significantly stable against decomposition.

### SUMMARY

The compositions of U.S. Pat. No. 6,045,826 such as those comprising PTS allow for the solubilization of lipophilic bioactives. However, for rigid molecules like CoQ₁₀ the emulsions that form lack desirable stability. In the present examples, the combination of lipophilic bioactive compounds such as CoQ₁₀ with oil (even in small amounts) was found to significantly improve emulsion stability. The emulsions were prepared by heating a composition comprising the bioactive lipophilic compound, the oil and the compound of Formula (I) (for example, the solubilizing agent PTS) to form a homogeneous melt, and combining the homogeneous melt with water using either blending or high shear mixing, optionally followed by homogenization by use of a microfluidizer, and rapid cool down, using cooling, ice, cold water, or a mixture of ice and water to obtain the emulsion.

Accordingly, the present application includes a composition comprising:
(i) a bioactive lipophilic compound;
(ii) an oil; and
(iii) at least one compound of Formula (I):

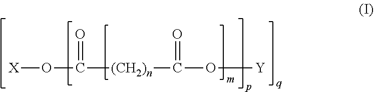

wherein
   X is a residue of a hydrophobic moiety selected from sterols, tocopherols and derivatives thereof;
   Y is a residue of a hydrophilic moiety selected from polyalcohols, polyethers and derivatives thereof;
   m is 0 or 1;
   n is an integer of from 0 to 18;
   p is 1 or 2; and
   q is 1 or 2.

The present application also includes an emulsion comprising a composition of the present application dispersed in water.

The present application also includes a pharmaceutical or cosmetic formulation comprising an emulsion of the present application and a biologically acceptable liquid carrier; and a dietary supplement comprising an emulsion of the present application and a biologically acceptable liquid carrier.

The present application also includes a beverage comprising the emulsion of the present application.

The present application also includes a use of an emulsion of the present application for the preparation of a pharmaceutical formulation or a cosmetic formulation; a use of an emulsion of the present application for the preparation of a dietary supplement; and a use of an emulsion of the present application for the preparation of a beverage.

The present application also includes a method for preparing an emulsion, the method comprising:
heating a composition of the present application to form a homogeneous melt; and
combining the homogeneous melt with water to obtain the emulsion.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The present application will now be described in greater detail with reference to the drawings in which:

FIG. 1 shows exemplary chromatograms obtained by liquid chromatography with evaporative light scattering detection (LC-ELSD) showing PTS composition of Table 1: PTS monomer (*) to PTS dimer (**) ratio (upper chromatogram) and impurities (lower chromatogram; A-I defined in Table 1).

FIG. 2 shows schematics of PTS monomer (upper structure) and PTS dimer (lower structure) relating to evaluation by ¹H-NMR in pyridine-ds.

FIG. 3 shows a plot of the stability of PTS-CoQ₁₀ exemplary emulsions prepared with different ratios of medium chain length triglyceride (MCT) oil after 20 weeks at 37° C., 10,000 psi pressure in a microfluidizer with 0, 1, 2 and 3 passes (scale: clear: 9-10; very slightly cloudy: 8; slightly cloudy: 7; cloudy: 5-6; very cloudy, precipitation: 1-4); temperature at 37° C.

FIG. 4 shows a plot of the turbidity of exemplary PTS-CoQ₁₀ emulsions prepared with different ratios of MCT oil when stored at 32° C.

FIG. 5 shows plots of the stability of micelle size of exemplary PTS-CoQ₁₀ emulsions: upper: 50% MCT oil microfluidizer, lower: 50% MCT oil, ice cooled (* line: 0 weeks, ** line: 12 weeks, 32° C., *** line: 12 months, RT).

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

As used in this application, the singular forms “a”, “an” and “the” include plural references unless the content clearly dictates otherwise.

The term “suitable” as used herein means that the selection of specific reagents or conditions will depend on the reaction being performed and the desired results, but nonetheless, can generally be made by a person skilled in the art once all relevant information is known.

The term “PTS monomer” as used herein refers to a compound having the following general structure:

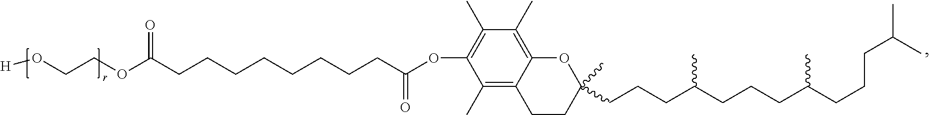

wherein r is 12 or 13.

The term “PTS dimer” as used herein refers to a compound having the following general structure:

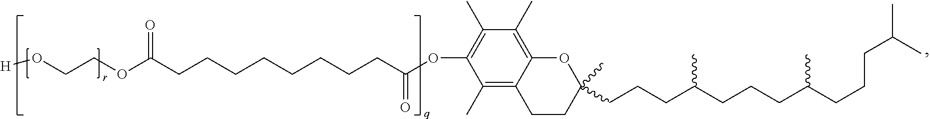

wherein r is 12 or 13; and q is 2.

The term “subject” as used herein includes all members of the animal kingdom including mammals, and suitably refers to humans.

The term “biologically acceptable” as used herein means compatible with the treatment of, administration to and/or use in subjects.

In embodiments of the application, the compounds described herein have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (e.g. less than 20%, suitably less than 10%, more suitably less than 5%) of compounds of the application having alternate stereochemistry.

The term “bioactive” as used herein refers to having biological activity. For example, a bioactive compound may have an effect on, cause a reaction in and/or trigger a response in living tissue such as a cell.

The term “lipophilic” as used herein refers to a compound that is soluble in fat-like solvents such as hydrocarbons.

The term “bioactive lipophilic compound” as used herein refers to a compound that is both bioactive and lipophilic.

The term “ubiquinone” as used herein refers to a compound also known as coenzyme Q (CoQ) having the following general structure:

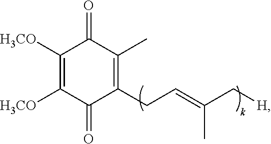

wherein k is an integer that denotes the number of isoprene subunits in the lipidic sidechain of ubiquinone. The number of isoprene subunits in the lipidic sidechain of a particular ubiquinone can also be referred to, for example, as the subscript k in the term “coenzyme Qₖ” or the term CoQₖ”. For example, the term “coenzyme Q₁₀” and the abbreviation “CoQ₁₀” refer to a ubiquinone having k=10. In an embodiment, k is an integer of from 6 to 10. In another embodiment, k is 10.

The term “ubiquinol” as used herein refers to a compound that is the fully reduced form of a ubiquinone and has the following general structure:

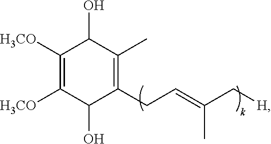

wherein k is an integer that denotes the number of isoprene subunits in the lipidic sidechain of ubiquinol. In an embodiment, k is an integer of from 6 to 10. In another embodiment of the present application, k is 10.

The term “lipophilic vitamin” as used herein refers to a lipophilic compound that is an essential micronutrient useful for the proper metabolic functioning of a subject. The term “vitamin” as used herein does not include minerals, essential fatty acids and essential amino acids. The term “essential” as used herein, for example, in reference to a micronutrient, fatty acid and/or amino acid means that the micronutrient, fatty acid and/or amino acid is not synthesized in the organism or is not synthesized in sufficient quantities for proper metabolic function. Lipophilic vitamins include vitamins A, D, E and K.

The term “lipophilic provitamin” as used herein refers to a lipophilic compound that is converted within a subject into a lipophilic vitamin. For example, β-carotene is a provitamin which can be converted into vitamin A.

The term “polyalcohol” as used herein refers a compound having the general formula HOCH₂(CHOH)ₓCH₂OH.

The term “polyether” as used herein refers to a compound that is an oligomer or polymer having repeating units comprising an ether functionality.

### II. Compositions and Uses

In the present examples, the combination of lipophilic bioactive compounds such as CoQ₁₀ with oil (even in small amounts) was found to significantly improve emulsion stability over emulsions not containing the oil.

Accordingly, the present application includes a composition comprising:
(i) a bioactive lipophilic compound;
(ii) an oil; and
(iii) at least one compound of Formula (I):

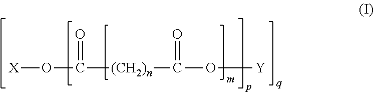

wherein
   X is a residue of a hydrophobic moiety selected from sterols, tocopherols and derivatives thereof;
   Y is a residue of a hydrophilic moiety selected from polyalcohols, polyethers and derivatives thereof;
   m is 0 or 1;
   n is an integer of from 0 to 18;
   p is 1 or 2; and
   q is 1 or 2.

In an embodiment, the bioactive lipophilic compound is selected from ubiquinones, ubiquinols, lipophilic vitamins, lipophilic provitamins, polyene macrolide antibiotics and combinations thereof. In an embodiment, the bioactive lipophilic compound is a ubiquinone. In another embodiment, the bioactive lipophilic compound is a ubiquinol. In a further embodiment, the bioactive lipophilic compound is a lipophilic vitamin. In another embodiment, the bioactive lipophilic compound is a lipophilic provitamin. In a further embodiment, the bioactive lipophilic compound is a polyene macrolide antibiotic. In another embodiment, the bioactive lipophilic compound is a combination of ubiquinones, ubiquinols, lipophilic vitamins, lipophilic provitamins and/or polyene macrolide antibiotics.

In an embodiment, the bioactive lipophilic compound is selected from coenzyme Q₁₀ (CoQ₁₀), lutein and β-carotene. In another embodiment, the bioactive lipophilic compound is CoQ₁₀. In a further embodiment, the bioactive lipophilic compound is lutein. In another embodiment of the present application, the bioactive lipophilic compound is β-carotene.

In an embodiment, the hydrophobic moiety in Formula (I) is selected from cholesterol, 7-dehydrocholesterol, campesterol, sitosterol, ergosterol, stigmasterol, α-tocopherol, β-tocopherol, γ-tocopherol and δ-tocopherol. In another embodiment, the hydrophobic moiety is α-tocopherol.

In an embodiment, the hydrophilic moiety in Formula (I) is a polyether. In another embodiment, the hydrophilic moiety is a polyalcohol. In an embodiment, the polyether is a polyalkylene glycol. In another embodiment, the polyalkylene glycol is a polyethylene glycol or a polypropylene glycol. In a further embodiment, the polyalkylene glycol is a polyethylene glycol. The term “polyalkylene glycol” as used herein includes polyalkylene glycols having an esterifiable hydroxy group at least at one end of the polymer as well as derivatives of such polymers having esterifiable carboxy groups. The residue of the hydrophilic moiety is the entire hydrophilic molecule, except for its esterified hydroxy or carboxy group or groups, such as a terminal hydroxy group of a polyethylene glycol. In an embodiment, the polyethylene glycol has an average molecular weight of from about 300 to about 5000. In another embodiment of the present application, the polyethylene glycol has an average molecular weight of from about 600 to about 1000.

In an embodiment, when p, m and q are all equal to 1 and the hydrophobic moiety is cholesterol, n is greater than 4 and not equal to 8. In another embodiment of the present application, when p, m and q are all equal to 1 and the hydrophobic moiety is α-(+)-tocopherol, n is not equal to 2.

In an embodiment, n is an integer of from 2 to 10. In another embodiment, n is an integer of from 6 to 10. In a further embodiment, n is 8.

In an embodiment, m is 1. In another embodiment, m is 0.

In an embodiment, p is 1. In another embodiment, p is 2.

In an embodiment, q is 1. In another embodiment, q is 2.

In an embodiment, the at least one compound of Formula (I) is polyoxyethanyl-α-tocopheryl sebacate (PTS) monomer, PTS dimer or combinations thereof. In another embodiment, the at least one compound of Formula (I) is PTS monomer. In a further embodiment, the at least one compound of Formula (I) is PTS dimer. In another embodiment, the at least one compound of Formula (I) is a combination of PTS monomer and PTS dimer.

In an embodiment, the ratio of the at least one compound of Formula (I) to the total amount (i.e. the sum of the amounts) of the bioactive lipophilic compound and the oil is from about 2:1 to about 12:1. In another embodiment, the ratio of the at least one compound of Formula (I) to the total amount of the bioactive lipophilic compound and the oil is about 3.5:1.

In one embodiment, the oil is any suitable oil. For example, it would be appreciated by the person skilled in the art that when the composition is for use in subjects, the oil is a biologically acceptable oil. In an embodiment, the oil is sunflower oil, medium chain length triglyceride (MCT) oil, long chain triglycerides (LCT) oil, hemp oil, vegetable oil, soybean oil, olive oil or combinations of two or more thereof. In another embodiment, the oil is sunflower oil. In a further embodiment, the oil is MCT oil.

In an embodiment, the oil is present in the composition in an amount of from about 1 wt % to about 98 wt %, based on the total weight of the oil and the bioactive lipophilic compound. In another embodiment, the oil is present in an amount of from about 20 wt % to about 60 wt %, based on the total weight of the oil and the bioactive lipophilic compound. In a further embodiment, the oil is present in an amount of from about 12 wt % to about 50 wt %, based on the total weight of the oil and the bioactive lipophilic compound. In another embodiment, the oil is present in an amount of from about 45 wt % to about 55 wt % or about 50 wt %, based on the total weight of the oil and the bioactive lipophilic compound.

The present application also includes an emulsion comprising a composition of the present application dispersed in water. It will be appreciated by a person skilled in the art that embodiments relating to the compositions in the emulsions of the present application can be varied as described herein in relation to the compositions of the present application.

In an embodiment, the composition is dispersed in the water in the form of micelles.

The concentration of the bioactive lipophilic compound and the oil in the emulsion can be any suitable concentration and may depend, for example, on the use of the emulsion and/or the identity of the bioactive lipophilic compound. In an embodiment, the concentration of the bioactive lipophilic compound and the oil (i.e. the sum of the amounts) is from about 1 mg/mL to about 100 mg/mL. In another embodiment, the concentration of the bioactive lipophilic compound and the oil is from about 25 mg/mL to about 55 mg/mL. In a further embodiment, the concentration of the bioactive lipophilic compound and the oil is from about 30 mg/mL to about 36 mg/mL or about 33 mg/mL. In another embodiment, the concentration of the bioactive lipophilic compound and the oil is from about 45 mg/mL to about 55 mg/mL or about 50 mg/mL. In some embodiments, for example, wherein the bioactive lipophilic compound is a ubiquinone or ubiquinol such as coenzyme Q₁₀, the concentration of the bioactive lipophilic compound is from about 15 mg/mL to about 30 mg/mL, about 15 mg/mL to about 18 mg/mL, about 16.5 mg/mL, about 20 mg/mL to about 30 mg/mL or about 25 mg/mL. In some embodiments, for example, wherein the bioactive lipophilic compound is a lipophilic vitamin or lipophilic provitamin such as lutein and/or β-carotene, the concentration of the bioactive lipophilic compound is from about 0.05 mg/mL to about 2 mg/mL, about 0.1 mg/mL to about 1 mg/mL or about 0.53 mg/mL.

In an embodiment, the emulsion further comprises one or more additives. In another embodiment, the additives are selected from adjuvants, colorants, flavoring agents, preservatives, buffers and combinations thereof.

The present application also includes a pharmaceutical or cosmetic formulation comprising an emulsion of the present application and a biologically acceptable carrier. The present application further includes a dietary supplement comprising an emulsion of the present application and a biologically acceptable carrier. The emulsions of the present application may advantageously provide a format for subjects that does not involve swallowing pills but instead offers a solution for the administration or use of bioactive lipophilic compounds in a liquid format. Accordingly, the present application also includes a pharmaceutical or cosmetic formulation comprising an emulsion of the present application and a biologically acceptable liquid carrier as well as a dietary supplement comprising an emulsion of the present application and a biologically acceptable liquid carrier.

In an embodiment, the pharmaceutical or cosmetic formulation is in the form of a spray, syrup or drop. In another embodiment of the present application, the dietary supplement is in the form of a spray, syrup or drop. A person skilled in the art would know how to prepare suitable formulations.

The present application also includes a beverage comprising the emulsion of the present application.

The present application also includes a use of an emulsion of the present application for the preparation of a pharmaceutical formulation or a cosmetic formulation. The present application further includes a use of an emulsion of the present application for the preparation of a dietary supplement. The present application also includes a use of an emulsion of the present application for the preparation of a beverage. For example, an emulsion of the present application can be added to any suitable beverage base such as water. In an embodiment, from about 1 mL to about 3 mL or about 2 mL of the emulsion is added per every 250 mL of the beverage base such as water.

### III. Methods

Emulsions that had significant stability over the corresponding emulsions that didn't contain an oil were prepared by heating a composition comprising a bioactive lipophilic compound, the oil and the compound of Formula (I) (for example, the solubilizing agent PTS) to form a homogeneous melt and combining the homogeneous melt with water using either blending or high shear mixing, optional homogenization by use of a microfluidizer and rapid cool down using cooling, ice, cold water, or a mixture of ice and water to obtain the emulsion.

Accordingly, the present application also includes a method for preparing an emulsion, the method comprising:
heating a composition of the present application to form a homogeneous melt; and
combining the homogeneous melt with water to obtain the emulsion.

It will be appreciated by a person skilled in the art that embodiments relating to the compositions in the methods for preparing an emulsion of the present application can be varied as described herein in relation to the compositions of the present application.

In an embodiment, the combining step comprises mixing the homogeneous melt and water at a temperature of from about 40° C. to about 95° C., for example, a temperature of from about 85° C. to about 90° C. for a time of about 15 minutes to about 4 hours, or about 150 minutes to about 210 minutes.

In one embodiment, the combining step comprises mixing the homogeneous melt with water using either blending or high shear mixing, optionally followed by homogenization by use of a microfluidizer, and rapid cool down, using cooling, ice, cold water, or a mixture of ice and water to obtain the emulsion

In some embodiments, subsequent to mixing, the method further comprises processing the mixture through a microfluidizer. The conditions for processing the mixture through a microfluidizer are any suitable conditions. In an embodiment, the conditions comprise a single pass through the microfluidizer. In another embodiment, the conditions comprise a pressure of from about 7,500 psi to about 12,500 psi, about 7,500 psi, about 10,000 psi or about 12,500 psi.

In an embodiment, the method further comprises cooling the mixture. It will be appreciated by the person skilled in the art that in embodiments comprising processing the mixture through a microfluidizer, the cooling can be subsequent or simultaneous to the processing of the mixture through the microfluidizer. The mixture is cooled to any suitable temperature. In an embodiment, the mixture is cooled to a temperature of about 1° C. to about 15° C. or about 4° C.

In some embodiments, the cooling comprises mixing the homogeneous emulsion with ice or a combination of water and ice. In an embodiment, the ratio by volume of water:ice in the final combination is about 2:1.

In an embodiment, the method further comprises filtering. The filter is any suitable filter, the selection of which can be made by the skilled person.

The following non-limiting examples are illustrative of the present application:

### EXAMPLES

### Example 1: Synthesis and Characterization of Polyoxyethanyl-α-Tocopheryl Sebacate (PTS)

The protocols for the synthesis of PTS have been developed on the lab bench scale and the synthesis has previously been demonstrated on a ton scale in 2009. It is a 2-step process using α-tocopherol, sebacoyl chloride and PEG 600. In an exemplary laboratory scale synthesis of PTS, a solution of 55 g (0.127 moles) α-tocopherol (T) and 23.6 mL (0.169 moles) triethylamine in 50 mL ethyl acetate were mixed at a temperature of about 0-5° C. and were added slowly over a period of 45 to 60 minutes to a solution of 0.170 moles sebacoyl chloride (S) in 50 mL ethyl acetate at about −15° C. and allowed to warm up to room temperature (about 20-25° C.) under stirring and stirred for an additional 60 min. The reaction mixture was diluted with 100 ml ethyl acetate and then slowly, over a period of about 60 minutes, added to a solution of 153.2 g (0.254 moles) PEG 600 (P) and 23.6 mL (0.169 moles) triethylamine in 120 mL ethyl acetate at room temperature (about 20-25° C.) and allowed to react for a time of about 60 min under stirring then subjected to a aqueous washes, concentrated, and subjected to organic washes.

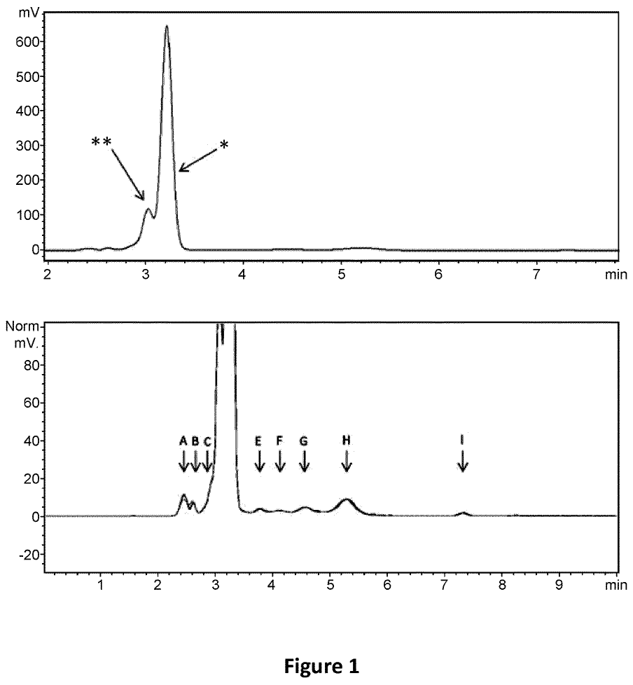

A more detailed analysis of the material from the commercial scale synthesis led to the putative identification of several impurities, as well as the discovery that PTS is in fact a mixture of PTS monomer and PTS dimer (the dimer having an additional sebacic acid and PEG group). The purity of PTS (monomer and dimer combined) is around 93% with the remaining 7% being synthesis side products and polymers (FIG. 1). In total, 8 impurities of one PTS sample were isolated and analysed using mass spectrometry (MS), nuclear magnetic resonance (NMR) spectroscopy, Fourier-transform infrared (FTIR) spectroscopy and ultraviolet-visible (UV) spectroscopy data. While not wishing to be limited by theory, the predicted structures have two or more components of the starting materials connected via ester bonds and in some cases forming an oligomer containing 2-4 sebacic acid-PEG units (Table 1).

TABLE 1
Summary of PTS impurities and putative structures.

| Retention | Amount | | |
|---|---|---|---|
| time [min] | [%] | Fraction | Putative structure |
| 2.4 | 1.30 ± 0.11 | PTS09A04-L-A | (PEG-Sebacate)₃ |
| 2.6 | 0.64 ± 0.08 | PTS09A04-L-B | Tocopheryl(Sebacate- |
| | | | PEG)₃ |
| 2.9 | | PTS09A04-L-C | Tocopheryl(Sebacate- |
| | | | PEG)₂ |
| 3.1 | 13.00 ± 0.22 | | PTS dimer (**) |
| 3.2 | 79.11 ± 0.29 | | PTS monomer (*) |
| 3.5 | | PTS09A04-L-D | insufficient material |
| 3.9 | 0.74 ± 0.01 | PTS09A04-L-E | Tocopheryl-(Sebacate- |
| | | | PEG)₃-Sebacate- |
| | | | Tocopheryl |
| 4.1 | 0.75 ± 0.03 | PTS09A04-L-F | Tocopheryl-(Sebacate- |
| | | | PEG)₂-Sebacate- |
| | | | Tocopheryl |
| 4.5 | 1.30 ± 0.08 | PTS09A04-L-G | Tocopheryl-(Sebacate- |
| | | | PEG)₂-Sebacate- |
| | | | Tocopheryl |
| 5.2 | 2.98 ± 0.05 | PTS09A04-L-H | Tocopheryl-(Sebacate- |
| | | | PEG)₁-Sebacate- |
| | | | Tocopheryl |
| 7.2 | 0.19 ± 0.01 | PTS09A04-L-I | Tocopheryl-Sebacate- |
| | | | Tocopheryl |

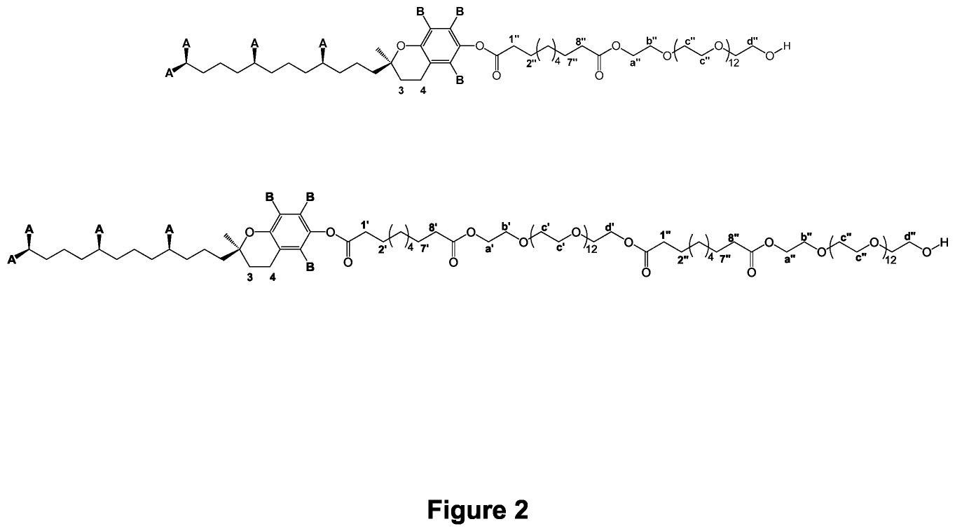

The ratio between PTS monomer to PTS dimer can be determined by either liquid chromatography using an evaporative light scattering detector LC-ELSD (FIG. 1) or by ¹H-NMR in pyridine-ds (FIG. 2) analysis. In pyridine-ds the terminal —CH₂—CH₂—OH group of the PEG subunit (d″ at 3.9 ppm) is separated from the internal —CH₂—CH₂—O— groups (b′, b″, c′ and c″ at 3.6 to 3.8 ppm) and —CH₂—CH₂—O—CO— groups (a′, a″ and d′ at 4.4 ppm), allowing for calculation of the ratio of PTS monomer to dimer. PTS contains an average of 73% PTS monomer according to NMR in pyridine-ds and an average of 65% PTS monomer according to LC-ELSD.

Further purification of the PTS monomer has been demonstrated in small lab scale experiments using additional washing steps (using acetonitrile and cyclohexane). PTS monomer was recovered from the cyclohexane fraction, while the ratio of PTS dimer to monomer increased in the acetonitrile fraction.

The amount of the PTS dimer compared to PTS monomer can also be increased by changes to the synthesis protocol by adjusting the ratios of the components and the order of the synthetic steps as well as by using Poly(ethylene glycol) methyl ether (m-PEG) instead of PEG.

### Example 2: Preparation of PTS Emulsions Containing Oils

PTS can solubilize numerous lipophilic bioactives, including CoQ₁₀ and nutritional oils. The stability of the resulting emulsions was found to be dependent on the substrate, the concentration, the substrate to PTS ratio and the addition of other ingredients that are present in the final formulation. The CoQ₁₀-PTS emulsion stability was found to be significantly susceptible to changes in the preparation of the emulsion and its use in final formulations.

The addition of oils (in this Example, sunflower oil or MCT oil (medium chain length triglycerides)) to the CoQ₁₀ in the preparation of the emulsions was tested first in a set of small batch scale experiments (up to 1 L). The emulsions were prepared using the following protocol: In a first step, the CoQ₁₀, the oil and the PTS formed a homogenous melt at a temperature of about 30-50° C., which was combined with hot water (about 65-95° C.) under high shear mixing. The resulting warm solution was homogenized using a microfluidizer testing different settings (different pressure settings and number of passes as set out in Tables 2 and 3). Incidentally, other carriers did not require emulsion formation using the high shear forces of a microfluidizer but can be done using a standard stirrer with mixing at high temperature; CoQ₁₀ is a much harder molecule to form stable micelles with. The resulting CoQ₁₀ emulsions were clear and the stability was tested by storage at elevated temperature (accelerated stability testing at 37° C.). All emulsions had a final substrate concentration (CoQ₁₀+oil) of 50 mg/ml with a PTS to substrate ratio of 3.5:1 by weight. Failing of the emulsions can be determined by an increasing turbidity/cloudiness of the solutions and the formation of a visible precipitate.

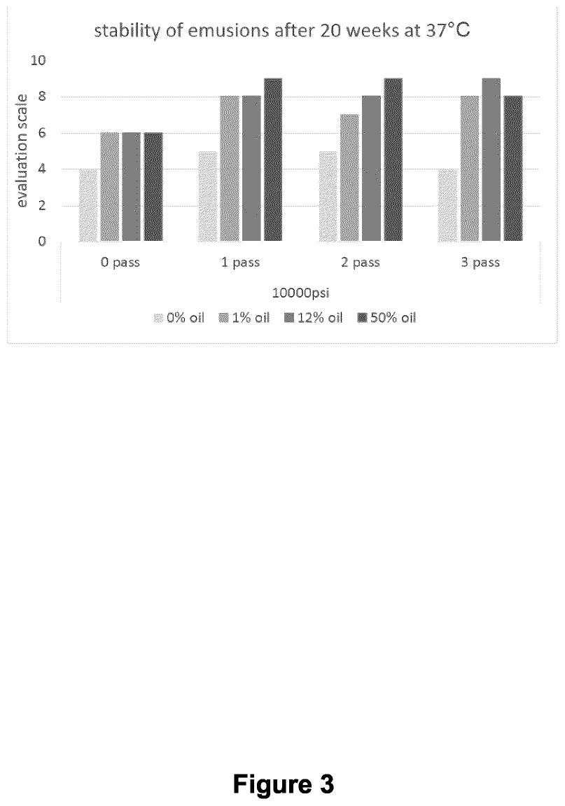

The resulting emulsions with oil concentrations of 1%, 12% and 50% were overall more stable in this accelerated shelf life stability study than the CoQ₁₀ emulsions without oil addition (see Table 2 for sunflower oil, 5 week stability data and Table 3 and FIG. 3 for MCT oil, 20 week stability). The data in Tables 2 and 3 uses the following evaluation scale: clear: 9-10; very slightly cloudy: 8; slightly cloudy: 7; cloudy: 5-6; very cloudy, precipitation: 1-4. A single pass was a preferred method for formation of CoQ₁₀ emulsions although the data of a second and third pass through the microfluidizer is shown.

TABLE 2
Stability of PTS-CoQ₁₀-sunflower oil emulsions prepared with
different ratios of sunflower oil at different pressures after 5 weeks
temperature at 37° C..

| Microfluidizer | | Sunflower oil (% of total substrate) | | | |
|---|---|---|---|---|---|
| conditions | | 0% | 1% | 12% | 50% |
| 3^{rd} | 7500 psi | 8 | 10 | 10 | 10 |
| pass | 10,000 psi | 8 | 9 | 10 | 10 |
| | 12,500 psi | 8 | 9 | 10 | 10 |

TABLE 3
Stability of PTS-CoQ₁₀-MCT emulsions prepared with different
ratios of MCT oil at different pressures after 20 weeks temperature at
37° C..

| Microfluidizer (MF) | | MCT oil (% of total substrate) | | | |
|---|---|---|---|---|---|
| conditions | | 0% | 1% | 12% | 50% |
| no MF | — | 4 | 6 | 6 | 6 |
| 7500 psi | 1^{st} pass | 5 | 8 | 8 | 9 |
| | 2^{nd} pass | 4 | 7 | 8 | 9 |
| | 3^{rd} pass | 4 | 7 | 8 | 8 |
| 10000 | 1^{st} pass | 5 | 8 | 8 | 9 |
| psi | 2^{nd} pass | 5 | 7 | 8 | 9 |
| | 3^{rd} pass | 4 | 8 | 9 | 8 |
| 12500 | 1^{st} pass | 5 | 8 | 8 | 8 |
| psi | 2^{nd} pass | 5 | 7 | 8 | 8 |
| | 3^{rd} pass | 4 | 8 | 7 | 8 |

In a second phase, the formulation of the emulsions was scaled up to 10 L and subsequently to a 60 L pilot manufacturing scale.

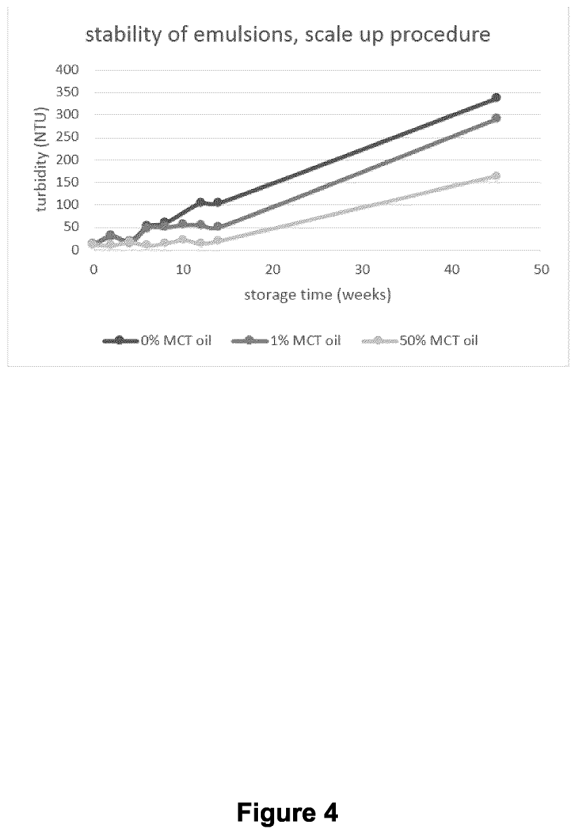

For the 10 L scale production, PTS-CoQ₁₀-MCT oil emulsions with a concentration of 50 mg substrate per ml with 0%, 1% and 50% of MCT oil were prepared with a PTS to substrate ratio of 3.5:1 by weight. CoQ₁₀, MCT oil and PTS were heated and mixed together to form a homogenous melt at a temperature of 40° C. to 60° C. Hot water (about 55 to 65° C.) was added or the mixture was heated with water to 60° C. and mixed at 3520 rpm for 20 to 30 minutes. The resulting solutions were processed through a microfluidizer at 10,000 psi, cooled to 4° C. over a period of about 1-5 seconds in the microfluidizer, and filtered through a 0.2 μm polyethylsulfone (PES) filter. The samples were stored at 32° C. for accelerated stability testing. The factor to compare accelerated shelf life testing at 32° C. to stability at room temperature has been previously determined to be 4 (1 week at 32° C. equals 1 month at room temperature). Samples were observed for colour, clarity, turbidity and precipitates every 2 weeks for 14 weeks. Additional samples were stored in a long term stability study at 32° C. for 45 weeks. Micelle size was analysed after 0 weeks and after 12 weeks. The results of the stability study are summarized in Table 4 and FIG. 4. The addition of the MCT oil had a significant effect on the stability of the emulsions measured by increasing turbidity and formation of visible precipitates. The addition of 1% MCT oil increased the stability slightly, the overall turbidity was lower starting around week 8 and the formation of large particles was delayed by 4 weeks from week 10 to week 14. An increase of the oil concentration to 50% resulted in a significant increase in stability with a lower turbidity starting in week 6 and no large precipitate particles were observed in the first 14 testing weeks.

TABLE 4
stability of PTS-CoQ₁₀-MCT emulsions prepared with different
ratios of MCT oil after 52 weeks at 32° C., progression of failing
emulsions from clear solutions to major precipitation.

| | Emulsion condition, precipitate formation | | | |
|---|---|---|---|---|
| | | | Larger | |
| MCT oil (% | Clear or | Very fine | particles, | Large |
| of total | very slightly | particles, | increased | particles, |
| substrate) | turbid | minor effect | effect | major effect |
| 50% MCT oil | Week 0 | Week 4 | Week 14 | Week 45 |
| 1% MCT oil | Week 0 | Week 2 | Week 8 | Week 14 |
| 0% MCT oil | Week 0 | Week 2 | Week 8 | Week 10 |

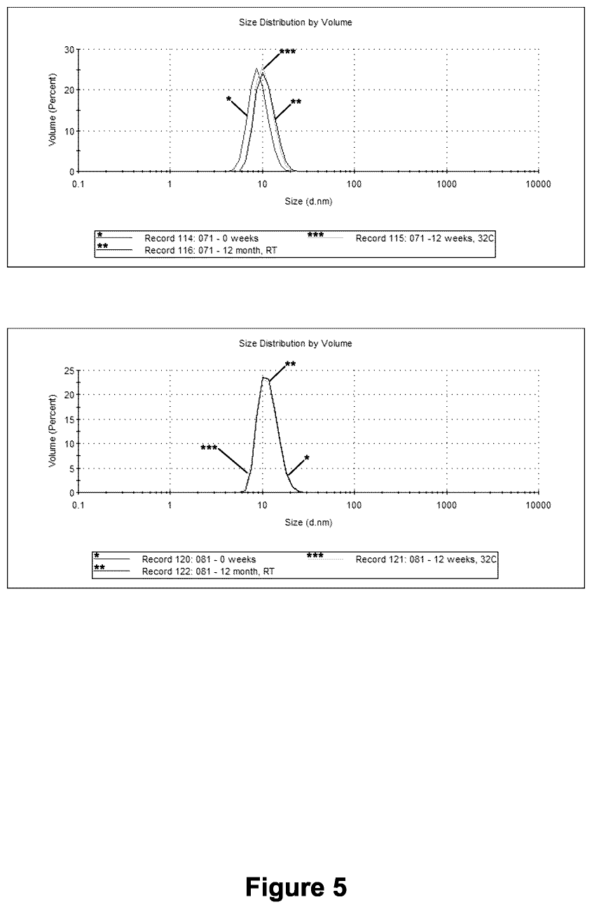

For the 60 L scale production, PTS-CoQ₁₀-MCT oil emulsions in 2 different concentrations with a PTS-substrate ratio of 3.5:1 by weight were produced using different protocols. First, a 50 mg substrate (CoQ₁₀+MCT oil) per ml with 50% of MCT oil emulsion was prepared as described above in the 10 L trial, except the heating temperature and mixing time was increased to 85° C. for 90 to 180 minutes. The resulting solutions were processed through a microfluidizer at 10,000 psi and cooled to 4° C. over a period of about 1-5 seconds, in the microfluidizer. Additional stock emulsions with a substrate concentration of 33 mg substrate (CoQ₁₀+MCT oil) per ml with 1%, 20% and 50% of MCT oil were prepared by mixing the mixture under heating as described above and adding one third of the total volume as ice to rapidly cool the solution under mixing, by about 70-90° C. over a period of 0.5 to 10 minutes. No microfluidizer was used for these samples. A natural preservative (Foodgard™, Biosecure Lab) was added to half the volume of all samples and they were filtered through 0.2 μm polyethylsulfone (PES) capsule filter. The samples were stored at 32° C. for accelerated stability testing and at room temperature for additional shelf life testing. Samples were observed for colour, clarity, turbidity and precipitates every 2 weeks for 24 weeks for samples at 32° C. and every 2 months for 12 months for samples at room temperature. The micelle size was analysed after 0 weeks, after 12 weeks at 32° C. and after 12 months at room temperature (about 20-25° C.). The results of the stability study are summarized in Table 5 and FIG. 5.

TABLE 5
stability of PTS-CoQ₁₀-MCT emulsions prepared with different
ratios of MCT oil after 52 weeks at 32° C., progression of failing
emulsions from clear solutions to major precipitation (MF:
microfluidizer).

| | Emulsion condition, precipitate formation | | | |
|---|---|---|---|---|
| | | | Larger | Large |
| MCT oil | Clear or | Very fine | particles, | particles, |
| (% of total | very slightly | particles, | increased | major |
| substrate) | turbid | minor effect | effect | effect |
| 50% MCT oil | Week 0 | Week 4 | Week 8 | Week 20 |
| MF | | | | |
| 50% MCT oil, | Week 0 | Week 8 | n.a. | n.a |
| ice | | | | |
| 20% MCT oil, | Week 0 | Week 8 | Week 12 | Week 12 |
| ice | | | | |
| 1% MCT oil, | Week 0 | Week 2 | Week 8 | Week 16 |
| ice | | | | |

As in the Examples described above, the addition of the MCT oil had a significant effect on the stability of the emulsions measured by increasing turbidity and formation of visible precipitate. This effect can also be observed in samples of emulsions that were not processed through a microfluidizer. Surprisingly, the samples cooled with ice and not processed through a microfluidizer cleared up considerably in the first few days of storage, whereas the turbidity dropped for most samples in the first couple of weeks. The biggest decrease in the turbidity happened in the first 24 h after preparing the solutions, with a slower decrease in the time after that. The micelle sizes for the ice cooled samples were all stable over the 12 week accelerated shelf life study period as well as over the 12 month shelf life stability study at room temperature.

The addition of 20% MCT oil increased the stability slightly compared to 1% MCT oil, the overall turbidity was lower and stable over 12 weeks, and the formation of large particles was delayed by 4 weeks from week 12 to week 16. An increase of the oil concentration to 50% resulted in a significant increase in stability with a lower turbidity in week 12 and the emulsions staying stable with only very small, fine particles observed over 24 h.

TABLE 6
stability of PTS-CoQ₁₀ emulsions prepared with different ratios
of MCT oil after 52 weeks at 32° C., progression of failing emulsions from
clear solutions to major precipitation (MF: microfluidizer) Turbidity and
micelle size measured on PTS-CoQ₁₀-MCT emulsions prepared with
different ratios of MCT oil over a 12 week period (MF: microfluidizer)

| | Emulsion condition: turbidity and micelle size | | | |
|---|---|---|---|---|
| MCT oil (% of | turbidity | | micelle size (DLS*) | |
| total substrate) | week 0 | week 12 | week 0 | week 12 |
| 50% MCT oil, MF | 40.5 NTU | 70.1 NTU | 9.89 nm | 11.3 nm |
| 50% MCT oil, ice | 214 NTU | 174 NTU | 12.68 nm | 12.73 nm |
| 20% MCT oil, ice | 207 NTU | 192 NTU | 11.27 nm | 12.53 nm |
| 1% MCT oil, ice | 221 NTU | 274 NTU | 10.11 nm | 10.71 nm |

*Dynamic light scattering.

Samples processed through the microfluidizer showed a minor decrease in turbidity over the first 24 h after manufacturing as well, but the effect was much lower than in the ice cooled samples. The samples were much clearer in the beginning with a smaller micelle size, with a higher percentage increase in turbidity and micelle size over 12 weeks, though the overall turbidity stayed below the values observed for the ice cooled samples over the whole monitoring period (Table 6). The microfluidizer treated samples developed large precipitate particles by week 20 (table 5). The emulsions processed through the microfluidizer had a slightly smaller micelle size with a less variation of the particle size, measured as the peak diameter (9.2 nm vs. 11.8 nm micelle size and 2.1 nm vs. 2.9 nm peak diameter). However, both the particle size and the peak diameter increased during the shelf life study period while the samples cooled with ice were more stable in both parameters.

TABLE 7
Micelle size and peak diameter measured on PTS-CoQ₁₀-MCT
emulsions over a 12 month period

| | | Micelle size | Peak |
|---|---|---|---|
| Sample | | (nm) | diameter (nm) |
| microfluidizer | 0 week | 9.2 ± 0.4 | 2.1 ± 0.18 |
| | 12 weeks 32° C. | 10.6 ± 0.2 | 2.4 ± 0.13 |
| | 12 month RT | 10.9 ± 0.4 | 2.6 ± 0.01 |
| ice | 0 week | 11.8 ± 0.2 | 2.9 ± 0.11 |
| | 12 weeks 32° C. | 11.6 ± 0.1 | 2.9 ± 0.04 |
| | 12 month RT | 11.8 ± 0.1 | 2.9 ± 0.09 |

Protocols for the formulations of PTS emulsions with substrates and oils with increased stability have been demonstrated. The oils include sunflower oil, MCT oil (medium chain triglycerides), LCT oil (long chain triglycerides), olive oil and hemp oil. The substrates include CoQ₁₀ as described hereinabove as well as carotenoids like β-carotene and lutein, Vitamin A.

### Example 3: Water Enhancer Products

The PTS stock emulsions with various substrates can be used as alternate delivery formats to swallowing pills for lipophilic bioactives to produce beverages or concentrates like water enhancer products or liquid supplements, sprays, syrups, drops, etc. The stability of the emulsions and products is directly correlated with the concentration of PTS and the substrate and the selection and concentration of additives, like preservatives, flavours, buffers, etc.

Water enhancer products commonly use around 2 mL of concentrate in a 250 mL glass of water. This makes it not only necessary to have concentrated emulsions to achieve desirable substrate concentrations in the final, diluted beverage, but also for those to be stable in the presence of high concentrated additives, e.g. flavours, citric acid, sweetener or sugar.

The production of the water enhancer products was scaled up from bench scale to pilot scale (proof of concept for up to 300 L). For the production of water enhancer products, stock emulsions prepared as described in example 2 were used. with CoQ₁₀, lutein, and β-Carotene as lipophilic compounds. A natural preservative (Foodgard™, Biosecure Lab) can also be added to the preparation. Water enhancers products were then prepared by diluting the stock emulsions with water under mixing and adding the additional components. The substrate concentrations in the stock emulsions and in the final water enhancer products are summarized in Table 8 and the general compositions of the water enhancer products are summarized in Table 9. Flavors were provided by International Flavors and Fragrances and colours provided by Frutarom.

TABLE 8
Concentrations of the substrates in both the stock emulsions
and the diluted water enhancer products.

| | PTS to | | |
|---|---|---|---|
| | Substrate | Stock Emulsion | Water Enhancer |
| Substrate | Ratio | Concentration | Concentration |
| CoQ₁₀ | 3.5 to 1 | 25.0 mg CoQ₁₀ | 3.125 mg CoQ₁₀ |
| 50% | | per mL | per mL |
| MCT | | 25.0 mg MCT | 3.125 mg MCT |
| Oil | | oil per mL | oil per mL |
| Lutein | 3 to 1 | 0.53 mg Lutein | 0.1 mg Lutein |
| MCT | (in oil) | per mL | per mL |
| Oil | | 26.7 mg MCT | 5 mg MCT |
| | | oil per mL | oil per mL |
| β- | 3 to 1 | 0.53 mg β-Carotene | 0.1 mg β-Carotene |
| Carotene | (in oil) | per mL | per mL |
| MCT Oil | | 26.7 mg MCT | 5 mg MCT |
| | | oil per mL | oil per mL |

TABLE 9
composition of water enhancer products: stock emulsion, flavor
and color addition dependent on selected substrates and flavor.

| Ingredient | Composition (%) |
|---|---|
| Water | 39.2 to 51.1 |
| Citric Acid | 21.00 |
| Sweetener (Stevia RA80) | 2.22 |
| Sodium Citrate | 1.50 |
| Preservative (Foodgard) | 0.30 |
| Stock emulsion | 12.5 to 18.75 |
| Flavour (IFF) | 11.25 to 16.89 |
| colours (Frutarom) | Combined 0.15 |

Accelerated shelf life testing at 32° C. and shelf life testing at room temperature (about 20-25° C.) was performed for the stock emulsions and the water enhancer products. The factor to compare accelerated shelf life testing at 32° C. to stability at room temperature has been previously determined to be 4 (1 week at 32° C. equals 1 month at room temperature). The stability was tested every 2 weeks for samples at 32° C. (for 24 weeks) and every 2 month for samples at room temperature (for 12 month). The physical characteristics (colour, clarity, turbidity, precipitates) and the taste profile (flavour and off-flavours, odour and off-odours) were monitored at each testing date for the concentrated product and the particle size by DLS were monitored after 0 weeks and 12 weeks at 32° C.

Several products prepared have a shelf life stability of at least 1 year. These products include β-carotene (antioxidant, peach, lemon or berry flavour), CoQ₁₀ (energy, mango or orange flavour) and lutein (berry flavour).

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A composition comprising:
(i) a bioactive lipophilic compound;
(ii) an oil; and
(iii) at least one compound of Formula (I): 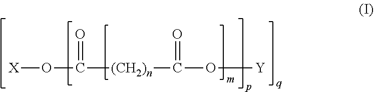
wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2.

2. The composition of claim 1, wherein the bioactive lipophilic compound is selected from ubiquinones, ubiquinols, lipophilic vitamins, lipophilic provitamins, polyene macrolide antibiotics and combinations thereof.

3. The composition of claim 2, wherein the bioactive lipophilic compound is selected from coenzyme Q₁₀ (CoQ₁₀), lutein and β-carotene.

4. The composition of claim 3, wherein the bioactive lipophilic compound is CoQ₁₀.

5. The composition of any one of claims 1 to 4, wherein the hydrophobic moiety is selected from cholesterol, 7-dehydrocholesterol, campesterol, sitosterol, ergosterol, stigmasterol, α-tocopherol, β-tocopherol, γ-tocopherol and δ-tocopherol.

6. The composition of claim 5, wherein the hydrophobic moiety is α-tocopherol.

7. The composition of any one of claims 1 to 6, wherein the hydrophilic moiety is a polyether.

8. The composition of any one of claims 1 to 7, wherein the polyether is a polyethylene glycol.

9. The composition of claim 8, wherein the polyethylene glycol has an average molecular weight of from about 300 to about 5000.

10. The composition of claim 8, wherein the polyethylene glycol has an average molecular weight of from about 600 to about 1000.

11. The composition of any one of claims 1 to 10, wherein n is 8.

12. The composition of any one of claims 1 to 11, wherein m is 1.

13. The composition of any one of claims 1 to 12, wherein p is 1.

14. The composition of any one of claims 1 to 13, wherein the at least one compound of Formula (I) is polyoxyethanyl-α-tocopheryl sebacate (PTS) monomer, PTS dimer or combinations thereof.

15. The composition of claim 14, wherein the at least one compound of Formula (I) is a combination of PTS monomer and PTS dimer.

16. The composition of any one of claims 1 to 15, wherein the ratio of the at least one compound of Formula (I) to the total amount of the bioactive lipophilic compound and the oil is from about 2:1 to about 12:1.

17. The composition of claim 16, wherein the ratio is about 3.5:1.

18. The composition of any one of claims 1 to 17, wherein the oil is sunflower oil, soybean oil, hemp oil, vegetable oil, olive oil, long chain triglycerides, medium chain length triglyceride (MCT) oil or combinations thereof.

19. The composition of any one of claims 1 to 18, wherein the oil is present in the composition in an amount of from about 1 wt % to about 98 wt %, based on the total weight of the oil and the bioactive lipophilic compound.

20. The composition of any one of claims 1 to 18, wherein the oil is present in the composition in an amount of from about 20 wt % to about 60 wt %, based on the total weight of the oil and the bioactive lipophilic compound.

21. An emulsion comprising the composition as defined in any one of claims 1 to 20 dispersed in water.

22. The emulsion of claim 21, wherein the composition is dispersed in the water in the form of micelles.

23. The emulsion of claim 21 or 22, wherein the concentration of the bioactive lipophilic compound and the oil is from about 0.1 mg/mL to about 100 mg/mL.

24. The emulsion of claim 23, wherein the concentration of the bioactive lipophilic compound and the oil is from about 25 mg/mL to about 55 mg/mL.

25. The emulsion of any one of claims 21 to 24, further comprising one or more additives.

26. The emulsion of claim 25, wherein the additives are selected from adjuvants, colorants, flavoring agents, preservatives, buffers, natural and artificial sweeteners and a combination of any two or more thereof.

27. A pharmaceutical or cosmetic formulation comprising the emulsion of any one of claims 21 to 26 and a biologically acceptable liquid carrier.

28. A dietary supplement comprising the emulsion of any one of claims 21 to 26 and a biologically acceptable liquid carrier.

29. The pharmaceutical or cosmetic formulation of claim 27 or the dietary supplement of claim 28 in the form of a spray, syrup or drop.

30. A beverage comprising the emulsion of any one of claims 21 to 26.

31. A use of an emulsion of any one of claims 21 to 26 for the preparation of a pharmaceutical formulation or a cosmetic formulation.

32. A use of an emulsion of any one of claims 21 to 26 for the preparation of a dietary supplement.

33. A use of an emulsion of any one of claims 21 to 26 for the preparation of a beverage.

34. A method for preparing an emulsion, the method comprising:
heating a composition as defined in any one of claims 1 to 20 to form a homogeneous melt; and
combining the homogeneous melt with water to obtain the emulsion.

35. The method of claim 34, wherein the combining comprises mixing the homogeneous melt and water at a temperature of from about 40° C. to about 95° C.

36. The method of claim 35, wherein subsequent to mixing, the method further comprises processing the mixture through a microfluidizer or homogenizer.

37. The method of claim 34 or 35, wherein the method further comprises cooling the mixture to a temperature below 20° C.

38. The method of claim 37, wherein the cooling comprises mixing the emulsion with ice or with a combination of water and ice.

39. The method of claim 37 or 38, wherein the cooling is at a rate of at least 5° C. per minute.

40. The method of any one of claims 34 to 39, further comprising filtering.
